(19) United States
(12) Patent Application Publication
(10) Pub. No.: US 2026/0290516 A1
(43) Pub. Date: Sep. 24, 2026

(54) METHOD FOR PREDICTING CHEMICAL REACTION USING PROJECTION OF ORBITAL COEFFICIENT VECTOR

(71) Applicant: Zhengzhou University, Zhengzhou (CN)

(72) Inventors: Donghui WEI, Zhengzhou (CN); Xiaofei SHI, Zhengzhou (CN); Yang WANG, Zhengzhou (CN); Zhipeng PEI, Zhengzhou (CN)

(21) Appl. No.: 19/380,204

(22) Filed: Nov. 5, 2025

(30) Foreign Application Priority Data
Nov. 5, 2024 (CN) 202411561963.9

(51) Int. Cl. G16C 20/10

(52) U.S. Cl. CPC G16C 20/10

(57) ABSTRACT

A method for predicting a chemical reaction using projection of an orbital coefficient vector is provided. The method includes calculating coefficient vector projections along multiple possible valence orbital overlap directions in a molecule by means of a developed valence orbital coefficient vector projection method, thereby obtaining reaction indices such as atomic vectorial reaction indices for different sites and directions. Through comparison of the atomic vectorial reaction indices for different sites, reactivity of a site can be predicted, that is, likelihood of an atom participating in a chemical reaction at that site. By calculating the atomic vectorial reaction indices, directional charge densities, and corresponding reaction energy barriers for different sites, likelihood of chemical reactions occurring at multiple potential reactive sites in the molecule can be quantitatively and directionally predicted.

### CROSS-REFERENCE TO RELATED APPLICATION

This patent application claims the benefit and priority to Chinese Patent Application No. 202411561963.9, filed with the China National Intellectual Performance Administration on Nov. 5, 2024, the disclosure of which is incorporated by reference herein in its entirety as part of the present application.

### TECHNICAL FIELD

The present disclosure relates to the technical field of theoretical and computational chemistry, and specifically to a method for predicting a chemical reaction using projection of an orbital coefficient vector.

### BACKGROUND

Under conditions such as lighting, heating, and addition of a catalyst, a chemical reaction can generally be divided into two stages: substrate activation and product conversion. In a substrate activation stage, an inert substrate can be activated into a highly active species; subsequently, in a stage of conversion to product, it is converted into a product through means such as coupling or addition reaction. Theoretical calculations can be used to investigate a new activation mechanism in the substrate activation stage, and to explain or even predict stereoselectivity, chemoselectivity, and regioselectivity of the reaction in the stage of conversion to product.

Chemical reactions are directional, so accurate prediction of reactivity requires consideration of a possible direction of orbital interactions. An ideal method for predicting reactivity should, as described in a chemical reaction mechanism, include a direction of electron transfer and be capable of determining a possible direction of orbital overlap. In fact, a type of molecular orbitals is closely related to the direction and pattern of orbital overlap and to a type of orbitals. For example, π orbitals typically undergo a pattern of side-by-side overlap perpendicular to a bond axis.

However, a currently commonly used reactivity index calculation method generally does not distinguish the direction and pattern of orbital overlap interactions. Although this can simplify calculations, it may be inaccurate when predicting reactivity of an atom possessing multiple orbital overlap directions within a molecule.

### SUMMARY

To address the problem existing in the prior art, the present disclosure provides a method for predicting a direction of a chemical reaction using projection of an orbital coefficient vector. The method provided in the present disclosure can be used to predict reactivity of an atom possessing multiple orbital overlap directions in a molecule, and thus predict likelihood of a chemical reaction.

To achieve the above object, the present disclosure provides the following technical solutions.

The present disclosure provides a method for predicting a direction of a chemical reaction using projection of an orbital coefficient vector. The method includes the following steps:
obtaining molecular orbitals based on a calculation result of a time-independent Schrödinger equation output by a quantum chemistry program such as Gaussian;
obtaining, based on the molecular orbitals, a linear combination of valence atomic orbitals 2px, 2py, and 2pz;
defining a projection direction of a main-group atom using a valence atomic orbital coefficient vector projection method;
calculating, based on the linear combination coefficients (i.e. C₂ₚₓ, C_{2py}, and C_{2pz}) of valence atomic orbitals 2px, 2py, and 2pz, projection of an orbital coefficient vector \vec{2p}(C₂ₚₓ, C_{2py}, C_{2pz}) onto a projection direction \vec{n}, to obtain a projection vector \vec{2p}′(C′₂ₚ_{ₓ}, C′₂ₚ_{_{y}}, C′₂ₚ_{_{z}}) of the main-group atom in a molecule onto the projection direction n′ and atomic vectorial reaction indices for different sites and directions;
obtaining a vectorial reactivity index F(\vec{n}) based on the projection vector of the main-group atom onto the projection direction \vec{n} and Equations 4 to 7; and
performing an iterative solution, based on the vectorial reactivity index F(\vec{n}), by using a gradient descent method to obtain a maximum vectorial reactivity index \vec{Fₘₐₓ}, for predicting likelihood of an atom participating in a chemical reaction at an associated site;
where the time-independent Schrödinger equation is given in Equation 1:

$$H {Ψ}_{n} = {E}_{n} {Ψ}_{n} , n = 1 , 2 … , \qquad Equation 1$$

where an expression for the molecular orbital is given in Equation 2:

$${Ψ}_{n} = \sum {C}_{i , n} {Ψ}_{i} , n = 1 , 2 … , \qquad Equation 2$$

where the linear combination of the valence atomic orbitals 2px, 2py, and 2pz is given in Equation 3:

$${Ψ}_{n} = … + {C}_{1 s} {ψ}_{1 s} + {C}_{2 s} {ψ}_{2 s} + \vec{2 p} ( {C}_{2 {p}_{x}} , {C}_{2 {p}_{y}} , {C}_{2 {p}_{z}} ) \cdot [ \begin{matrix}{{ψ}_{2 p}}_{x} \\ {ψ}_{2 {p}_{y}} \\ {ψ}_{2 {p}_{z}}\end{matrix} ] + … , n = 1 , 2 … , \qquad Equation 3$$

$${D}_{μ v}^{{p}_{1 / 2}} = {η}_{i} {\sum }_{i} {C}_{μ i}^{{p}_{1 / 2}} {C}_{vi}^{{p}_{1 / 2}} \qquad Equation 4$$

$${P}_{A B}^{′} = {\sum }_{a ∈ A} {\sum }_{b ∈ B} {( {D}^{{p}_{1}} S )}_{a b} {( {D}^{{p}_{1}} S )}_{b a} \qquad Equation 5$$

$${M}_{A B}^{′} = {\sum }_{a ∈ A} {\sum }_{b ∈ B} {( {D}^{{p}_{2}} S )}_{a b} {( {D}^{{p}_{2}} S )}_{b a} \qquad Equation 6$$

$$\vec{{F}_{A}} = [ {M}_{max} - {\sum }_{B} {M}_{A B}^{′} ] \cdot \vec{n} \qquad Equation 7$$

where in Equations 4-7,

$${D}_{μ v}^{{p}_{1 / 2}}$$

is a density matrix corresponding to a modified coefficient matrix with the replacement of (C₂ₚ_{ₓ}, C₂ₚ_{_{y}}, and C₂ₚ_{_{z}}) by

$$( {C}_{2 {p}_{x}}^{′} , {C}_{2 {p}_{y}}^{′} , and {C}_{2 {p}_{z}}^{′} ) ;$$

$${M}_{A B}^{′}$$

is a constrained Mayer bond order between an atom A and all its neighboring atoms; \vec{F_{A}} is a vectorial reactivity index of the atom A; and

$${P}_{A B}^{′}$$

is a constrained π bond order between the atom A and all its neighboring atoms.

Preferably, the linear combination is expanded in a product from the coefficient vector \vec{2p}(C₂ₚ_{ₓ}, C₂ₚ_{_{y}}, C₂ₚ_{_{z}}) and a matrix of corresponding atomic orbitals.

Preferably, for a planar unsaturated main-group atom, defined projection directions are maintained at equal angles, i.e., with an angle around a bond≥90°.

Preferably, for a carbene-type unsaturated main-group atom, a defined projection direction is distributed within a semicircular region.

Preferably, for a linear unsaturated main-group atom, a defined projection direction is distributed within a circular region.

The present disclosure makes it possible to calculate coefficient vector projections onto multiple possible valence orbital overlap directions in a molecule by means of a developed valence orbital coefficient vector projection method, thereby obtaining reaction indices such as atomic vectorial reaction indices for different sites and directions. Through comparison of the atomic vectorial reaction indices for different sites, reactivity of each site can be predicted, that is, likelihood of an atom participating in a chemical reaction at that site. By calculating the atomic vectorial reaction indices, directional atomic charge, and corresponding reaction energy barriers for different sites, likelihood of chemical reactions occurring at multiple potential reactive sites in the molecule can be quantitatively and directionally predicted. This has potential application value in precise synthesis for addressing issues such as reaction reactivity prediction and chemoselectivity control.

### BRIEF DESCRIPTION OF THE DRAWINGS

FIG. 1A shows a flowchart of a valence orbital coefficient vector projection method according to the present disclosure;

FIG. 1B shows a schematic diagram of a defined possible projection direction for a planar unsaturated main-group atom;

FIG. 1C shows a schematic diagram of defined possible projection directions for a linear unsaturated main-group atom;

FIG. 1D shows a schematic diagram of defined possible projection directions for a carbene-type unsaturated main-group atom;

FIG. 2A shows calculation of a π bond order

$$( {P}_{A B}^{′} )$$

using projection vectors \vec{2p′} (atom A) and \vec{2p′} (atom B);

FIG. 2B shows calculation of a constrained Mayer bond order

$$( {M}_{A B}^{′} )$$

using the \vec{2p′} (atom A);

FIG. 3A shows bond lengths (Å) for different conformations obtained by rotating a dihedral angle Φ(C2-C3-C12-C14) of a biphenyl molecule at a B3LYP/6-31G(d,p) level; and

FIG. 3B shows π bond orders and Mayer bond orders calculated using the valence orbital coefficient vector projection method with respect to different conformations obtained by rotating a dihedral angle Φ(C2-C3-C12-C14) of a biphenyl molecule at a B3LYP/6-31G(d,p) level;

FIG. 4A and FIG. 4B show prediction of a maximum vectorial reactivity index \vec{Fₘₐₓ} and an atomic vectorial reaction index using the valence orbital coefficient vector projection method.

### DETAILED DESCRIPTION OF THE EMBODIMENTS

The present disclosure provides a method for predicting a direction of a chemical reaction using projection of an orbital coefficient vector (POCV) method. The method includes the following steps:
obtaining molecular orbitals based on a calculation result of a time-independent Schrödinger equation, output by a quantum chemistry program such as Gaussian;
obtaining, based on the molecular orbitals, a linear combination of valence atomic orbitals 2px, 2py, and 2pz;
defining a projection direction of a main-group atom using a valence atomic orbital coefficient vector projection method;
calculating, based on the linear combination of valence atomic orbitals 2px, 2py, and 2pz, projection of an orbital coefficient vector onto a projection direction \vec{n}, to obtain a projection vector [symbol] of a main-group atom in the molecule onto the projection direction \vec{n} and atomic vectorial reaction indices for different sites and directions;
obtaining a vectorial reactivity index F(\vec{n}) based on the projection vector of the main-group atom onto the projection direction \vec{n} and Equations 4 to 7; and
performing an iterative solution, based on the vectorial reactivity index F(\vec{n}), by using a gradient descent method to obtained a maximum vectorial reactivity index \vec{Fₘₐₓ}, for predicting likelihood of an atom participating in a chemical reaction at an associated site.

According to the present disclosure, the molecular orbitals are obtained based on the calculation result of the time-independent Schrödinger equation, output by a quantum chemistry program such as Gaussian.

The time-independent Schrödinger equation is given in Equation 1:

$$H {Ψ}_{n} = {E}_{n} {Ψ}_{n} , n = 1 , 2 … \qquad Equation 1$$

where His a Hamiltonian matrix, Ψₙ is a n-th molecular orbital, and Eₙ is energy of the n-th molecular orbital.

An expression for the molecular orbital is given in Equation 2:

$${Ψ}_{n} = \sum {C}_{i , n} {Ψ}_{i} , n = 1 , 2 … \qquad Equation 2$$

wherein Ψᵢ is an i-th atomic orbital; and C_{i,n} is a coefficient of the i-th atomic orbital in the n-th molecular orbital.

According to the present disclosure, the linear combination of the valence atomic orbitals 2px, 2py, and 2pz is obtained based on the molecular orbitals.

As an implementation, the linear combination is an expanded in a product form of a coefficient vector

$$\vec{2 p} ( {C}_{2 {p}_{x}} , {C}_{2 {p}_{y}} , {C}_{2 {p}_{z}} )$$

and a matrix of corresponding atomic orbitals, i.e., Equation 3:

$${Ψ}_{n} = … + {C}_{1 s} {ψ}_{1 s} + {C}_{2 s} {ψ}_{2 s} + \vec{2 p} ( {C}_{2 {p}_{x}} , {C}_{2 {p}_{y}} , {C}_{2 {p}_{z}} ) \cdot [ \begin{matrix}{{ψ}_{2 p}}_{x} \\ {ψ}_{2 {p}_{y}} \\ {ψ}_{2 {p}_{z}}\end{matrix} ] + … , n = 1 , 2 … \qquad Equation 3$$

wherein, ψ₁ₛ, ψ₂ₛ, ψ₂ₚ_{ₓ}, ψ₂ₚ_{_{y}} and ψ₂ₚ_{_{z}} denote atomic orbitals 1s, 2s, 2px, 2py and 2pz, respectively; C₁ₛ, C₂ₛ, C₂ₚ_{ₓ}, C₂ₚ_{_{y}} and C₂ₚ_{_{z}}, denote coefficients of corresponding atomic orbitals; and \vec{2p}(C₂ₚ_{ₓ}, C₂ₚ_{_{y}}, C₂ₚ_{_{z}}) represents the orbital coefficient vector composed of three coefficients C₂ₚ_{ₓ}, C₂ₚ_{_{y}} and C₂ₚ_{_{z}};

The projection direction of the unsaturated main-group atom is defined using the valence atomic orbital coefficient vector projection method.

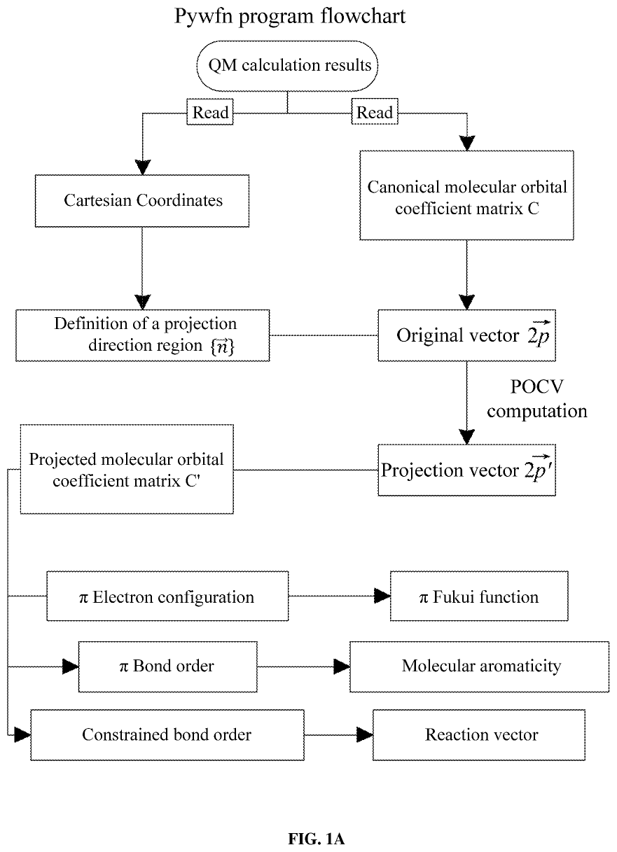

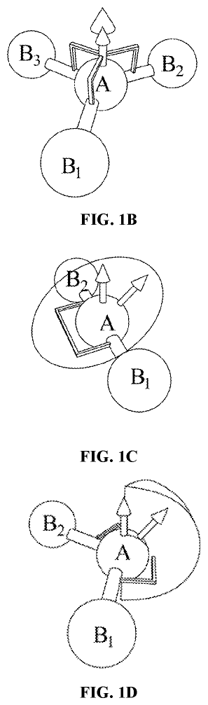

As an implementation, as shown in FIG. 1B-FIG. 1D, for a planar unsaturated main-group atom, defined projection directions are maintained at equal angles with respect to each of the chemical bonds between the unsaturated main-group atom and adjacent atoms, i.e., the angle between a bond and the defined projection direction≥90°; for a carbene-type unsaturated main-group atom, a defined projection direction is those projection directions distributed within a semicircular region; and for a linear unsaturated main-group atom, a defined projection directions is those projection directions distributed throughout a circular region.

The projection of the orbital coefficient vector onto the projection direction \vec{n} is calculated based on the linear combination of valence atomic orbitals 2px, 2py, and 2pz, to obtain the projection vector [symbol] of the main-group atom in the molecule onto the projection direction n′ and the atomic vectorial reaction indices for different sites and directions.

As an implementation, projection of the orbital coefficient vector \vec{2p}(C₂ₚ_{ₓ}, C₂ₚ_{_{y}}, C₂ₚ_{_{z}}) of a main-group atom (denoted as A), onto a projection direction \vec{n} yields a new vector

$${\vec{2 p}}^{′} ( {C}_{2 {p}_{x}}^{′} , {C}_{2 {p}_{y}}^{′} , {C}_{2 {p}_{z}}^{′} ) ,$$

as expressed in Equation 8:

$${\vec{2 p}}^{′} ( {C}_{2 {p}_{x}}^{′} , {C}_{2 {p}_{y}}^{′} , {C}_{2 {p}_{z}}^{′} ) = \frac{\vec{2 p} \cdot \vec{n}}{\vec{n}} \times \frac{\vec{n}}{\vert \vec{n} \vert} . \qquad Equation 8$$

The vectorial reactivity index F(\vec{n}) is obtained based on the projection vector of the main-group atom onto the projection direction \vec{n} and Equations 4 to 7;

$${D}_{μ v}^{{p}_{1 / 2}} = {η}_{i} {\sum }_{i} {C}_{μ i}^{{p}_{1 / 2}} {C}_{vi}^{{p}_{1 / 2}} , \qquad Equation 4$$

where in Equations 4,

$${D}_{μ v}^{{p}_{1 / 2}}$$

is a density matrix corresponding to a modified coefficient matrix; μi represents the element in the μ-th row and i-th column of the coefficient matrix, and vi represents the element in the v-th row and i-th column of the coefficient matrix; p₁ and p₂ respectively denote the projection schemes for calculating the π bond order and the constrained bond order, specifically, p₁ denotes using the normal vector of atom A for normal vector of atom B while ignoring the coefficients of other atoms; p₂ denotes retaining only the coefficient of atom A, keeping the coefficients of other atoms unchanged, and preserving the coefficient of the s-orbital; ηᵢ denotes a number of electrons occupied on the i-th molecular orbital;

$${P}_{A B}^{′} = {\sum }_{a ∈ A} {\sum }_{b ∈ B} {( {D}^{{p}_{1}} S )}_{a b} {( {D}^{{p}_{1}} S )}_{b a} , \qquad Equation 5$$

$${M}_{AB}^{′} = {\sum }_{a ∈ A} {\sum }_{b ∈ B} {( {D}^{{p}_{2}} S )}_{a b} {( {D}^{{p}_{2}} S )}_{b a} , \qquad Equation 6$$

$$\vec{{F}_{A}} = [ {M}_{{max}^{-}} {\sum }_{B} {M}_{AB}^{′} ] \cdot \vec{n} , \qquad Equation 7$$

where in Equations 5 to 7,

$${P}_{A B}^{′}$$

is a constrained π bond order between the atom A and all its neighboring atoms;

$${M}_{AB}^{′}$$

is a constrained Mayer bond order between an atom A and all its neighboring atoms; S is an overlap matrix; AB represents the bond between atom A and atom B; a and b are the basis function indices belonging to atom A and atom B, respectively; ab represents the row index belonging to atom A and the column index belonging to atom B, while ba represents the row index belonging to atom B and the column index belonging to atom A; \vec{F_{A}} is a vectorial reactivity index of the atom A; and Mₘₐₓ is a maximum value of the constrained Mayer bond order.

The iterative solution is performed by using the gradient descent method based on the vectorial reactivity index F(\vec{n}) to obtain the maximum vectorial reactivity index \vec{Fₘₐₓ}, for predicting the likelihood of the atom participating in the chemical reaction at the associated site.

During calculation of F(\vec{n}), a maximum bond order of a carbon atom along a single direction is defined as 3.

Iteration stops when F(\vec{n}) reaches a maximum value or a condition is not satisfied, and a final vector \vec{Fₘₐₓ} is obtained. It should be noted that there are three initial guesses for the projection direction \vec{n}, corresponding to the three types of atoms shown in FIG. 1B-FIG. 1D. When the atom A has three neighboring atoms (i.e., B1, B2, and B3), an initial projection direction \vec{n₀} is a normal vector of a plane formed by these three neighboring atoms; when the atom A has two neighboring atoms (B1 and B2) and the atom A, B1 and B2 are collinear, the initial projection direction \vec{n₀} is any direction perpendicular to this line, in which \vec{n₀} is a random unit vector; and when the atom A has two neighboring atoms (B1 and B2) and the atom A, B1 and B2 are non-collinear, the initial projection direction \vec{n₀} is a sum of two vectors from the atom A to the neighboring atoms (B1 and B2).

In the present disclosure, it is also possible to calculate coefficient vector projections onto multiple possible valence orbital overlap directions in a molecule by means of the valence orbital coefficient vector projection method, thereby obtaining the reaction indices such as atomic vectorial reaction indices for different sites and directions.

In the present disclosure, correctness of a predicted result is further verified by calculating a reaction energy barrier. If a predicted reactive site is indeed a site where a reaction occurs, a corresponding reaction energy barrier is relatively low, resulting in a relatively high reaction rate; conversely, if the predicted site is not an active site, the corresponding reaction energy barrier is relatively high, resulting in a relatively low reaction rate.

In the present disclosure, development of a corresponding program for wavefunction analysis and visualization is further included. Programming with Python may effectively improve the efficiency of extracting orbital information from an output file of quantum chemical calculation software (such as Gaussian) and calculating vectorial reaction indices, such as vectorial reaction indices, by means of the valence orbital coefficient vector projection method, and reduce the risk of errors.

In addition, the present disclosure also utilizes the Python language to further develop and optimize the program so as to realize characteristic orbital visualization, graphical display and processing of electronic structure information and directional reaction indices, thereby providing convenience and support for construction of a large-scale molecular reactivity database in the future.

In the present disclosure, construction of a reactivity index database for functional molecules, such as carbene-type and axially chiral pyridoxal-type molecules is further included. In addition to constructing a large library of molecular structures, such as carbene and axially chiral pyridoxine, the present disclosure can integrate publicly available organic molecule datasets, such as QM9, to enhance diversity and practical utility of the database. In the database, in addition to properties such as π-electron distribution and a reactivity index, chemical structure information, a physicochemical property, and the like of a molecule are included, so as to more comprehensively describe characteristics of the molecule.

In summary, in the present disclosure, a new orbital overlap direction-based strategy for predicting a chemical reaction is established by calculating atomic vectorial reaction indices, directional charge densities, and corresponding reaction energy barriers for different sites. Using the valence orbital coefficient vector projection method, a corresponding program for wavefunction analysis, computation, and visualization is developed, thereby enabling accurate prediction of different types of chemical reactions of an atom in a molecule.

FIG. 1A shows a flowchart of a valence orbital coefficient vector projection method according to the present disclosure. FIG. 1B shows a schematic diagram of a defined projection direction for a planar unsaturated main-group atom. FIG. 1C shows a schematic diagram of defined possible projection directions for a linear unsaturated main-group atom. FIG. 1D shows a schematic diagram of defined possible projection directions for a carbene-type unsaturated main-group atom.

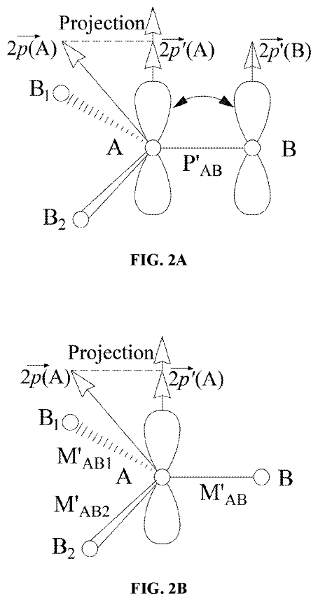

FIG. 2A-FIG. 2B shows (a) calculation of a π bond order

$$( {P}_{A B}^{′} )$$

using projection vectors \vec{2p′}(A) and \vec{2p′}(B); and (b) calculation of a constrained Mayer bond order

$$( {M}_{AB}^{′} )$$

using the \vec{2p′}(A), where the vectors \vec{2p′}(A) and \vec{2p′}(B) are projections of the vectors \vec{2p}(A) and \vec{2p}(B) onto their respective projection directions \vec{n} (e.g., a direction of a plane normal vector, which is always perpendicular to an existing bond).

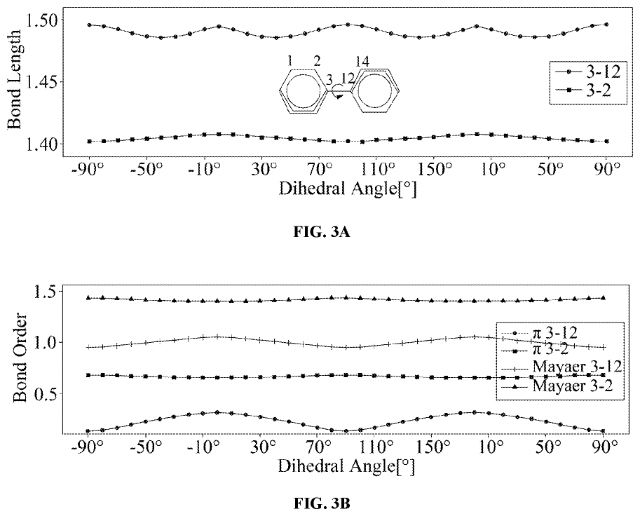

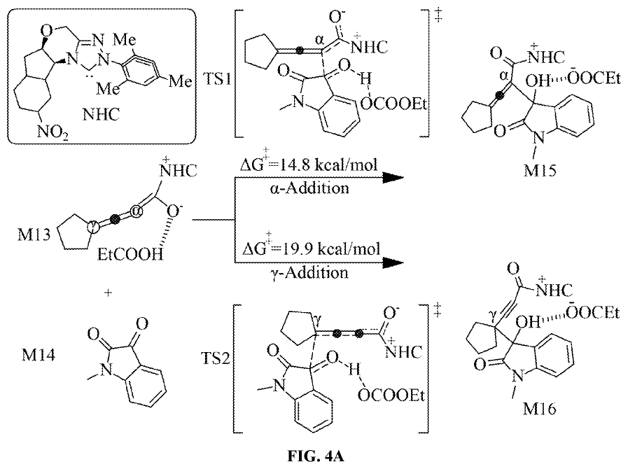

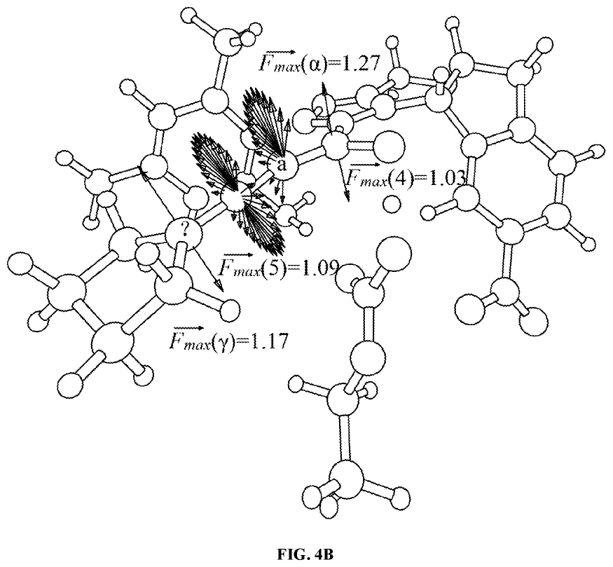

FIG. 3A-FIG. 3B shows (a) bond lengths (Å) and (b) π bond orders and Mayer bond orders calculated using the valence orbital coefficient vector projection method for different configurations and conformations obtained by rotating a dihedral angle Φ (C2-C3-C12-C14) of a biphenyl molecule at a B3LYP/6-31G (d,p) level. FIG. 4A-FIG. 4B shows prediction of an atomic vectorial reaction index using the valence orbital coefficient vector projection method and comparison leading to a maximum vectorial reactivity index \vec{Fₘₐₓ}.

The technical solutions provided by the present disclosure will be described in detail in conjunction with the embodiments below, which cannot be construed as limiting the scope of protection of the present disclosure.

### Embodiment 1

As shown in FIG. 3A-FIG. 3B, a biphenyl molecule is first selected and then a three-dimensional structure thereof is constructed, and 36 structures are obtained by rotating a dihedral angle C2-C3-C12-C14 and performing a rigid scan in 10° increments. For each structure, a calculation result of a time-independent Schrödinger equation, output by a quantum chemistry program such as Gaussian (Equations 1-3), is used to obtain molecular orbitals, which are composed of a linear combination of corresponding valence atomic orbitals 2px, 2py, and 2pz.

The time-independent Schrödinger equation is given in Equation 1:

$$H {Ψ}_{n} = {E}_{n} {Ψ}_{n} , n = 1 , 2 … \qquad Equation 1$$

An expression for the molecular orbital is given in Equation 2:

$${Ψ}_{n} = \sum {C}_{i , n} {Ψ}_{i} , n = 1 , 2 … \qquad Equation 2$$

The linear combination of the valence atomic orbitals 2px, 2py, and 2pz is given in Equation 3:

$${Ψ}_{n} = … + {C}_{1 s} {ψ}_{1 s} + {C}_{2 s} {ψ}_{2 s} + \vec{2 p} ( {C}_{2 {p}_{x}} , {C}_{2 {p}_{y}} , {C}_{2 {p}_{z}} ) \cdot [ \begin{matrix}{ψ}_{2 {p}_{x}} \\ {ψ}_{2 {p}_{y}} \\ {ψ}_{2 {p}_{z}}\end{matrix} ] + … , n = 1 , 2 … \qquad Equation 3$$

The projection vectors

$${\vec{2 p}}^{′} ( {C}_{2 {p}_{x}}^{′} , {C}_{2 {p}_{y}}^{′} , {C}_{2 {p}_{z}}^{′} )$$

of the orbital coefficient vectors \vec{2p}(C₂ₚ_{ₓ}, C₂ₚ_{_{y}}, C₂ₚ_{_{z}}), onto the projection direction \vec{n} are selected for calculation according to the atomic types in FIG. 1B-FIG. 1D and Equation 8.

$${\vec{2 p}}^{′} := ( {C}_{2 {p}_{x}}^{′} , {C}_{2 {p}_{y}}^{′} , {C}_{2 {p}_{z}}^{′} , ) = \frac{\vec{2 p} \cdot \vec{n}}{\vec{n}} \times \frac{\vec{n}}{\vert \vec{n} \vert} \qquad Equation 8$$

Subsequently, by replacing the original coefficients C₂ₚₓ, C_{2py}, C_{2pz} with the new coefficients

$${C}_{2 px}^{′} , {C}_{2 py}^{′} , {C}_{2 pz}^{′} ,$$

a new coefficient matrix C′ is obtained. A density matrix

$${D}_{μ v}^{{p}_{1 / 2}}$$

is then calculated using Equation 4, and the corresponding π bond orders are calculated according to Equation 5. A variation curve of the π bond orders is plotted, as shown in FIG. 3B.

The Mayer bond orders can be directly obtained by substituting the original coefficient matrix C into the density matrix and a Mayer bond order calculation formula, which are in the same form as Equations 4 and 5.

$${D}_{μ v}^{{p}_{1 / 2}} = {η}_{i} {\sum }_{i} {C}_{μ i}^{{p}_{1 / 2}} {C}_{vi}^{{p}_{1 / 2}} , \qquad Equation 4$$

$${P}_{A B}^{′} = {\sum }_{a ∈ A} {\sum }_{b ∈ B} {( {D}^{{p}_{1}} S )}_{a b} {( {D}^{{p}_{1}} S )}_{b a} . \qquad Equation 5$$

As can be seen from FIG. 3B, the variation curve of the π bond orders calculated by this method is smoother and exhibits stability comparable to that of the Mayer bond orders. Moreover, an absolute value of a π bond order for C3-C12 is significantly more reasonable than a value obtained by subtracting 1 from a Mayer bond order for C3-C12, which would result in a negative value. It should be noted that the molecular orbitals change continuously during dihedral angle scan, and thus the bond orders should also vary continuously. The orbital projection method is able to capture π-electron components in the molecular orbitals, which are also continuous and smooth in variation. In contrast, the conventional π-orbital selection method cannot handle σ-π orbital separation for a non-planar molecule. Therefore, the orbital projection method is more reasonable than the π-orbital selection method.

### Embodiment 2

As shown in FIG. 4A and FIG. 4B, an reactivity intermediate is first selected and then a three-dimensional structure thereof is constructed, and molecular orbitals are obtained based on a calculation result of a time-independent Schrödinger equation, output by a quantum chemistry program such as Gaussian (Equations 1 to 3), where the molecular orbitals are composed of a linear combination of corresponding valence atomic orbitals 2px, 2py, and 2pz.

The time-independent Schrödinger equation is given in Equation 1;

$$H {Ψ}_{n} = {E}_{n} {Ψ}_{n} , n = 1 , 2 … \qquad Equation 1$$

an expression for the molecular orbital is given in Equation 2;

$${Ψ}_{n} = \sum {C}_{i , n} {Ψ}_{i} , n = 1 , 2 … \qquad Equation 2$$

the linear combination of the valence atomic orbitals 2px, 2py, and 2pz is given in Equation 3;

$${Ψ}_{n} = … + {C}_{1 s} {ψ}_{1 s} + {C}_{2 s} {ψ}_{2 s} + \vec{2 p} ( {C}_{2 {p}_{x}} , {C}_{2 {p}_{y}} , {C}_{2 {p}_{z}} ) \cdot [ \begin{matrix}{ψ}_{2 {p}_{x}} \\ {ψ}_{2 {p}_{y}} \\ {ψ}_{2 {p}_{z}}\end{matrix} ] + … , n = 1 , 2 … \qquad Equation 3$$

The projection vectors

$${\vec{2 p}}^{′} ( {C}_{2 {p}_{x}}^{′} , {C}_{2 {p}_{y}}^{′} , {C}_{2 {p}_{z}}^{′} )$$

the orbital coefficient vectors \vec{2p}(C₂ₚ_{ₓ}, C₂ₚ_{_{y}}, C₂ₚ_{_{z}}), onto the projection direction n are selected for calculation according to the atomic types in FIG. 1B-FIG. 1D and Equation 8.

$${\vec{2 p}}^{′} := ( {C}_{2 {p}_{x}}^{′} , {C}_{2 {p}_{y}}^{′} , {C}_{2 {p}_{z}}^{′} ) = \frac{\vec{2 p} \cdot \vec{n}}{\vec{n}} \times \frac{\vec{n}}{\vert \vec{n} \vert} \qquad Equation 8$$

Subsequently, by replacing the original coefficients C₂ₚₓ, C_{2py}, C_{2pz} with the new coefficients

$${C}_{2 px}^{′} , {C}_{2 py}^{′} , {C}_{2 pz}^{′} ,$$

a new coefficient matrix C′ is obtained. A density matrix

$${D}_{μ v}^{{p}_{1 / 2}} ,$$

is then calculated using Equation 4, and the corresponding constrained bond order M′_{AB} and a vectorial reaction index F(\vec{n}) onto a selected projection direction are calculated according to Equations 6 and 7. Finally, as shown in FIG. 4A and FIG. 4B, the maximum vectorial reactivity index \vec{Fₘₐₓ} may be obtained through a comparison of a vectorial reactivity index F(\vec{n}) of three carbon atoms at three reactive sites along different directions. In FIG. 4A and FIG. 4B, the calculation are performed for three possible reaction sites (i.e., C(α), C(β), and C(γ)) of the reactive intermediate. After comparison, it was found that the maximum reaction vector is 1.27 at the C(α) site. Therefore, the C(α) site is predicted to be the most likely reaction site.

To verify the above argument, reaction energy barriers for α-addition and γ-addition are further calculated. The reaction energy barrier (14.8 kcal/mol) for α-addition is lower than that for γ-addition (19.9 kcal/mol), which also confirms that the likely reactive site is the C(α) site. In addition, the calculation results are consistent with experimental observations, indicating the reliability of this method in predicting chemoselectivity of a reaction.

$${D}_{μ v}^{{p}_{1 / 2}} = {η}_{i} {\sum }_{j} {C}_{μ i}^{{p}_{1 / 2}} {C}_{vi}^{{p}_{1 / 2}} , \qquad Equation 4$$

$${M}_{A B}^{′} = {\sum }_{a ∈ A} {\sum }_{b ∈ B} {( {D}^{{p}_{2}} S )}_{a b} {( {D}^{{p}_{2}} S )}_{b a} , \qquad Equation 6$$

$$\vec{{F}_{A}} = [ {M}_{{max}^{-}} {\sum }_{B} {M}_{A B}^{′} ] \cdot \vec{n} . \qquad Equation 7$$

Although the embodiments described above have provided a detailed description of the present disclosure, they are only a part of, rather than all of the embodiments of the present disclosure. Other embodiments can be obtained according to the embodiments of the present disclosure without creative efforts, and all such embodiments shall fall within the scope of protection of the present disclosure.

1. A method for predicting a direction of a chemical reaction using projection of an orbital coefficient vector (POCV) method, comprising following steps:
obtaining molecular orbitals based on a calculation result of a time-independent Schrödinger equation output by a quantum chemistry program;
obtaining, based on the molecular orbitals, a linear combination of valence atomic orbitals 2px, 2py, and 2pz;
defining a projection direction of a main-group atom using a valence atomic orbital coefficient vector projection method;
calculating, based on the linear combination of valence atomic orbitals 2px, 2py, and 2pz, projection of an orbital coefficient vector onto a projection direction \vec{n}, to obtain a projection vector of the main-group atom in a molecule onto the projection direction \vec{n} and atomic vectorial reaction indices for different sites and directions;
obtaining a vectorial reactivity index F(\vec{n}) based on the projection vector of the main-group atom onto the projection direction \vec{n} and Equations 4 to 7; and
performing an iterative solution, based on the vectorial reactivity index F(\vec{n}), by using a gradient descent method to obtain a maximum vectorial reactivity index \vec{Fₘₐₓ}, for predicting likelihood of an atom participating in a chemical reaction at an associated site;
wherein the time-independent Schrödinger equation is given in Equation 1:
$$H {Ψ}_{n} = {E}_{n} {Ψ}_{n} , n = 1 , 2 … , \qquad Equation 1$$
wherein H is a Hamiltonian matrix, Ψₙ is a n-th molecular orbital, and Eₙ is energy of the n-th molecular orbital;
an expression for the molecular orbital is given in Equation 2:
$${Ψ}_{n} = \sum {C}_{i , n} {Ψ}_{i} , n = 1 , 2 … , \qquad Equation 2$$
wherein Ψᵢ is an i-th atomic orbital; and C_{i,n} is a coefficient of the i-th atomic orbital in the n-th molecular orbital;
the linear combination of the valence atomic orbitals 2px, 2py, and 2pz is given in Equation 3:
$${Ψ}_{n} = … + {C}_{1 s} {ψ}_{1 s} + {C}_{2 s} {ψ}_{2 s} + \vec{2 p} ( {C}_{2 {p}_{x}} , {C}_{2 {p}_{y}} , {C}_{2 {p}_{z}} ) \cdot [ \begin{matrix}{ψ}_{2 {p}_{x}} \\ {ψ}_{2 {p}_{y}} \\ {ψ}_{2 {p}_{z}}\end{matrix} ] + … , n = 1 , 2 … , \qquad Equation 3$$
wherein, ψ₁ₛ, ψ₂ₛ, ψ₂ₚ_{_{y}} and ψ₂ₚ_{_{z}} denote atomic orbitals 1s, 2s, 2px, 2py and 2pz, respectively; C₁ₛ, C₂ₛ, C₂ₚ_{ₓ}, C₂ₚ_{_{y}}, and C₂ₚ_{_{z}} denote coefficients of corresponding atomic orbitals; and \vec{2p}(C₂ₚ_{ₓ}, C₂ₚ_{_{y}}, C₂ₚ_{_{z}}) represents the orbital coefficient vector composed of three coefficients C₂ₚ_{ₓ}, C₂ₚ_{_{y}} and C₂ₚ_{_{z}};
$${D}_{μ v}^{{p}_{1 / 2}} = {η}_{i} {\sum }_{i} {C}_{μ i}^{{p}_{1 / 2}} {C}_{v i}^{{p}_{1 / 2}} , \qquad Equation 4$$
$${P}_{A B}^{′} = {\sum }_{a ∈ A} {\sum }_{b ∈ B} {( {D}^{{p}_{1}} S )}_{a b} {( {D}^{{p}_{1}} S )}_{b a} , \qquad Equation 5$$
$${M}_{A B}^{′} = {\sum }_{a ∈ A} {\sum }_{b ∈ B} {( {D}^{{p}_{2}} S )}_{a b} {( {D}^{{p}_{2}} S )}_{b a} , \qquad Equation 6$$
$$\vec{{F}_{A}} = [ {M}_{{max}^{-}} {\sum }_{B} {M}_{A B}^{′} ] \cdot \vec{n} , \qquad Equation 7$$
wherein in Equations 4-7, p₁ and p₂ denote the POCV method for a π bond order and the POCV method for a Mayer bond order, respectively;
$${D}_{μ v}^{{p}_{1 / 2}}$$
is a density matrix corresponding to a modified coefficient matrix;
$${C}_{μ i}^{{p}_{1 / 2}} and {C}_{v i}^{{p}_{1 / 2}}$$
are coefficient matrices; ηᵢ denotes a number of electrons occupied on the i-th molecular orbital; S is an overlap matrix;
$${M}_{AB}^{′}$$
is a constrained Mayer bond order between an atom A and its neighboring atom B; a and b are basis function indices belonging to the atom A and the atom B, respectively; ab represents a row index belonging to the atom A and a column index belonging to the atom B, while ba represents the row index belonging to the atom B and the column index belonging to the atom A; \vec{F_{A}} is a vectorial reactivity index of the atom A; and
$${P}_{AB}^{′}$$
is a constrained π bond order between the atom A and all its neighboring atoms; Mₘₐₓ is a maximum value of the constrained Mayer bond order.

2. The method according to claim 1, wherein the linear combination is expanded in a product form of the orbital coefficient vector \vec{2p}(C₂ₚ_{ₓ}, C₂ₚ_{_{y}}, C₂ₚ_{_{z}}) and a matrix of corresponding atomic orbitals.

3. The method according to claim 1, wherein for a planar unsaturated main-group atom, a defined projection direction is maintained at equal angles with respect to each of bonds between the planar unsaturated main-group atom and neighboring atoms of the planar unsaturated main-group atom, wherein each angle is greater than or equal to 90°.

4. The method according to claim 1, wherein for a carbene-type unsaturated main-group atom, a defined projection direction is defined as forming an angle equal to or greater than 90° with respect to each bond between the carbene-type unsaturated main-group atom and neighboring atoms of the carbene-type unsaturated main-group atom.

5. The method according to claim 1, wherein for a linear unsaturated main-group atom, a defined projection direction points in any direction perpendicular to a line connecting the linear unsaturated main-group atom and two neighboring atoms of the linear unsaturated main-group atom.
